(19) United States
(12) Patent Application Publication
(10) Pub. No.: US 2015/0327815 A1
(43) Pub. Date: Nov. 19, 2015

(54) BIOSIGNAL MEASURING DEVICE

(71) Applicant: Roemsystem Corp., Daejeon-si (KR)

(72) Inventor: JUNG JIN HWANG, Sejong-si (KR)

(21) Appl. No.: 14/706,950

(22) Filed: May 7, 2015

(30) Foreign Application Priority Data
May 13, 2014 (KR) 10-2014-0057107

(51) Int. Cl. A61B 5/00; A61B 5/053

(52) U.S. Cl. CPC A61B 5/7214; A61B 5/053; A61B 5/7225; A61B 5/7278

(57) ABSTRACT

According to an embodiment of the present disclosure, a biosignal measuring device comprises two channels respectively detecting bio-potential signals from a human body through two electrodes, a biosignal extracting unit including two detecting means respectively amplifying the bio-potential signals through amplifiers, respectively, differentially operating the amplified bio-potential signals through a differential operator, and selectively adopting respective output signals of the two detecting means to obtain a biosignal, and an impedance correcting means adjusting the amplitude of the amplifiers of the two detecting means so that the power of a common mode noise signal caused by an impedance imbalance between the channels and included in the obtained biosignal is reduced to suppress the common mode noise signal.

### CROSS-REFERENCE TO RELATED APPLICATIONS

This patent application claims priority under 35 U.S.C. §119 to Korean Patent Application No. 10-2014-0057107, filed on May 13, 2014, in the Korean Intellectual Property Office, the disclosure of which is incorporated by reference herein in its entirety

### TECHNICAL FIELD

Embodiments of the present disclosure relate to devices for measuring biosignals, and more specifically, to biosignal measuring devices that, even when an impedance imbalance occurs, may exactly extract noise-suppressed biosignal waveforms through signal correction.

### DISCUSSION OF RELATED ART

Biosignals are tiny electrical signals that occur between human cells and are used in medical fields. Examples of biosignals include electrocardiograms, electromyograms, and brain waves.

Upon measuring biosignals from a human body, noise may be introduced into the biosignals, rendering precise measurement difficult.

A biosignal measuring device is shown in FIG. 1. The biosignal measuring device includes two electrodes 10 and 20 placed on particular parts of a human body, where biosignals are sensed, a biosignal extracting unit 40 receiving bio-potential signals through conductive wires 11 and 21 respectively connected to the two electrodes 10 and 20 and performing a differential operation to obtain a biosignal, and a signal processor 50 storing, analyzing, and transmitting wirelessly or wiredly the obtained biosignal. The biosignal measuring device may further include a driven right leg circuit (DRL) 60 and a reference electrode 30 placed on a particular part of the human body, and the biosignal measuring device extracts common mode noise signals from the signal output from the biosignal extracting unit 40, amplifies the common mode noise signals through an inverting amplifier Gr, and applies the inverting-amplified common mode noise signals to the reference electrode 30 through the conductive wire 31.

The bio-potential signals measured through the two electrodes 10 and 20, i.e., two channels, include relatively high-level external common mode noise signals of the same phase in addition to tiny biosignals. The external noise signals are of the same phase and are simultaneously detected by the two electrodes 10 and 20. The external noise signals may be suppressed by the differential operation of the biosignal extracting unit 40, and thus, the biosignals may be obtained.

A representative common mode noise signal is a noise signal that is introduced from a commercial power source Vs. The commercial electricity is of 60 Hz in Korea and 50 Hz in Europe. Accordingly, the noise signal induced by the commercial electricity is a 60 Hz noise signal in Korea and a 50 Hz noise signal in Europe.

Although the biosignal measuring device shown in FIG. 1 includes only one measuring unit consisting of two electrodes 10 and 20, a plurality of measuring units may be connected to a multiplexer (MUX) that selectively connects a particular one of the unit measurers to the biosignal extracting unit 40. Korean Patent Application Publication 10-2012-0102444 discloses a configuration for extracting a biosignal using a plurality of measuring units.

When the impedances between the electrodes and the human body are varied to cause a difference in impedance between the two electrodes, common mode noise signals significantly larger as compared with the biosignal to be measured might not be suppressed by the differential operation of the biosignal extracting unit 40. In other words, if an impedance imbalance between the two electrodes arises, common mode noise signals much larger than the biosignal might not be sufficiently suppressed by the differential operation, significantly deteriorating the accuracy of biosignal measurement.

To address such issue, Korean Patent No. 10-0868071 discloses a method for monitoring impedances of electrodes to detect an impedance imbalance between the electrodes.

However, impedance imbalance may be frequent as the human body moves. Further, in the case where electrodes are attached onto the human body using electrolyte gels, impedance imbalance may arise due to differences in hardness between the electrolyte gels. Performing impedance balancing whenever impedance imbalance occurs may be very burdensome and incorrect.

### SUMMARY

According to an embodiment of the present disclosure, a biosignal measuring device comprises two channels respectively detecting bio-potential signals from a human body through two electrodes, a biosignal extracting unit including two detecting means respectively amplifying the bio-potential signals through amplifiers, respectively, differentially operating the amplified bio-potential signals through a differential operator, and selectively adopting respective output signals of the two detecting means to obtain a biosignal, and an impedance correcting means adjusting the amplitude of the amplifiers of the two detecting means so that the power of a common mode noise signal caused by an impedance imbalance between the channels and included in the obtained biosignal is reduced to suppress the common mode noise signal.

The impedance correcting means alternately selects the two detecting means, adjusts the amplitude of an amplifier of a selected detecting means until the amplitude of the amplifier of the selected detecting means is smaller than the amplitude of an amplifier of an unselected detecting means, and when the power of the common mode noise signal reaches a predetermined convergent condition, adopts, as the biosignal, a signal output from one of the two detecting means producing a relatively smaller common mode noise signal.

The impedance correcting means starts to adjust the amplitude of the amplifier of the selected detecting means after the amplitude of the amplifier of the selected detecting means reaches the amplitude of the amplifier of the unselected detecting means.

The biosignal measuring device further comprises a remaining noise suppressing means summing signals output from two amplifiers included in one of the two detecting means of the biosignal extracting unit to obtain a common mode noise signal, normalizing the obtained common mode noise signal to the power of the common mode noise signal included in the biosignal obtained by the biosignal extracting unit, and subtracting the normalized common mode noise signal from the biosignal obtained by the biosignal extracting unit to obtain a biosignal where a remaining common mode noise signal has been suppressed.

After the impedance correcting means is operated, the remaining noise suppressing means is operated to suppress the remaining common mode noise signal after suppressing the common mode noise signal that occurs due to the impedance imbalance between the channels.

One of the channels is connected to an amplifier connected to a negative input terminal of a differential operator of a first detecting means of the two detecting means and an amplifier connected to a positive input terminal of a differential operator of a second detecting means of the two detecting means, and the other of the channels is connected to an amplifier connected to a negative input terminal of the differential operator of the second detecting means of the two detecting means and an amplifier connected to a positive input terminal of the differential operator of the first detecting means of the two detecting means. The biosignal extracting unit includes a differential operating unit differentially operating signals output from the two detecting means to obtain a biosignal. The impedance correcting means adjusts the respective amplifiers of the two detecting means, respectively connected to the channels, so that the power of a common mode noise signal included in the biosignal obtained by the differential operating unit is reduced.

The differential operating unit of the biosignal extracting unit normalizes the signals output from the two detecting means so that common mode noise signals included in the signals output from the two detecting means are substantially the same in power and then differentially operates the normalized output signals to obtain the biosignal.

The impedance correcting means previously designates a polarity of one of input terminals of the differential operator of one of the two detecting means and adjusts amplitude so that the amplitude of an amplifier connected to the input terminal of the designated polarity is larger than the amplitude of an amplifier connected to another input terminal of the differential operator.

The power of the common mode noise signal includes any one of power of a commercial electricity frequency component, power of a signal with a predetermined frequency applied to the human body, and power of a signal with a predetermined pattern.

According to an embodiment of the present disclosure, a biosignal measuring device comprises a first amplifier connected with a first channel, a second amplifier connected with a second channel, a third amplifier configured to differentially operate respective outputs of the first amplifier and the second amplifier to output a noise-suppressed signal, and a impedance corrector configured to adjust the amplitude of the first amplifier and the second amplifier to reduce a common mode noise signal included in the noise-suppressed-signal.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a circuit view illustrating a biosignal measuring device according to a related art;

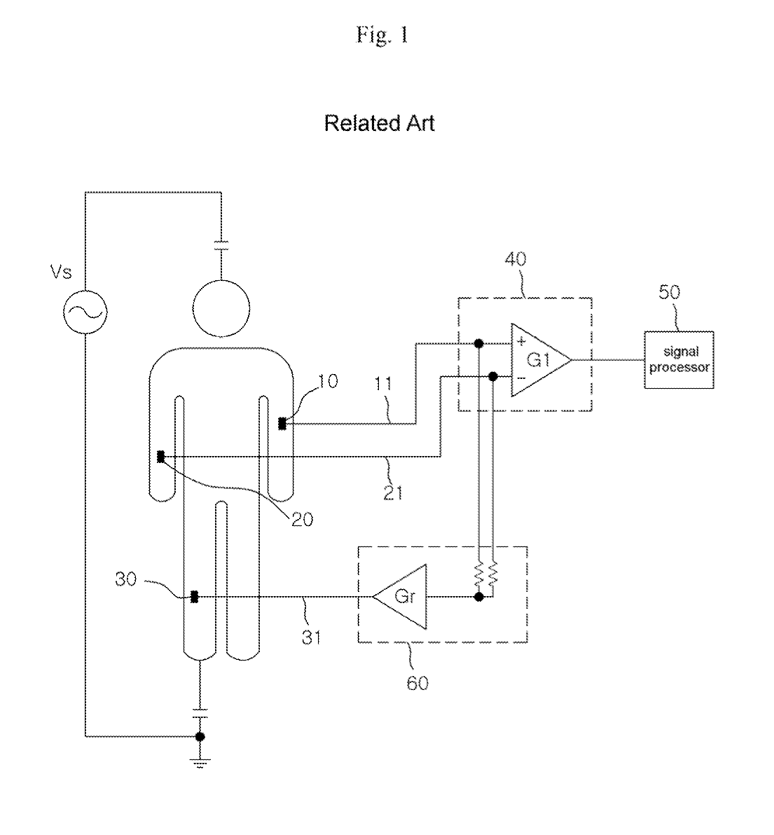

FIG. 2 is a circuit view illustrating a biosignal measuring device according to an embodiment of the present disclosure;

FIG. 3 is an equivalent circuit view illustrating the biosignal measuring device shown in FIG. 2, according to an embodiment of the present disclosure;

FIG. 4 is an equivalent circuit view illustrating a biosignal measuring device according to an embodiment of the present disclosure;

FIG. 5 is an equivalent circuit view illustrating a biosignal measuring device according to an embodiment of the present disclosure; and

FIG. 6 is an equivalent circuit view illustrating a biosignal measuring device according to an embodiment of the present disclosure.

### DETAILED DESCRIPTION OF EXEMPLARY EMBODIMENTS

Hereinafter, exemplary embodiments of the inventive concept will be described in detail with reference to the accompanying drawings. The inventive concept, however, may be modified in various different ways, and should not be construed as limited to the embodiments set forth herein. The same reference denotations may be used to refer to the same or similar elements throughout the specification and the drawings. It will be understood that when an element or layer is referred to as being “on” or “connected to” another element or layer, it can be directly on or connected to the other element or layer, or intervening elements or layers may be present. As used herein, the singular forms “a,” “an,” and “the” are intended to include the plural forms as well, unless the context clearly indicates otherwise.

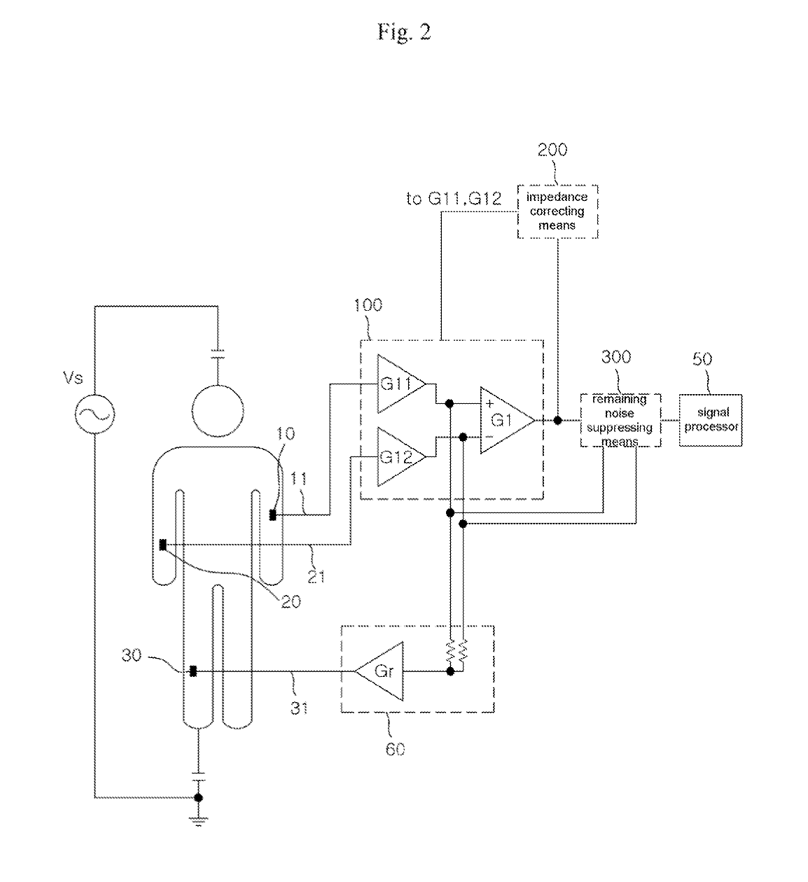

FIG. 2 is a circuit view illustrating a biosignal measuring device according to an embodiment of the present disclosure.

Referring to FIG. 2, the biosignal measuring device includes a plurality of electrodes 10 and detecting bio-potential signals on the skin in particular parts of a human body that are designated depending on the type of a biosignal to be measured; a biosignal extracting unit 100 obtaining a common mode noise-suppressed biosignal by performing a differential operation on a bio-potential signals detected by two of the plurality of electrodes; and a signal processor 50 configured to store, analyze, and transmit, wiredly or wirelessly, the obtained biosignal.

The biosignal extracting unit 100 includes two amplifiers G11 and G12 that respectively amplifies the bio-potential signals input through the two electrodes, and the bio signal extracting unit 100 performs a differential operation on the amplified bio-potential signals.

The biosignal measuring device further includes an impedance correcting means 200 adjusting the amplitude of the amplifiers G11 and G12 to reduce the power of common mode noise signals remaining in the biosignal obtained by the biosignal extracting unit 100; and a remaining noise suppressing means 300 that further suppresses common mode noise signals remaining in the biosignal obtained by the biosignal extracting unit 100 in addition to the noise suppression by the impedance correcting means 200 and transfers the noise-suppressed biosignal to the signal processor 50.

The plurality of electrodes 10 and 20 may directly contact the human skin and are electrically connected with the biosignal extracting unit 100 via conductive wires 11 and 21, respectively. Although two electrodes 10 and 20 are illustrated in FIG. 2, this is a mere example, and according to embodiments, three or more electrodes may be used, e.g., to further measure electromyograms, electrocardiograms, and brainwaves or electroencephalograms.

In case two or more electrodes are used, any of the electrodes may be selectively connected to the biosignal extracting unit 100 using a multiplexer (MUX) or switch. Alternatively, multiple biosignal extracting units 100 may be connected to the plurality of electrodes, respectively, measuring all of the signals detected by the electrodes. The connections between the electrodes and the biosignal extracting unit(s) 100 may be varied depending on applications for measuring biosignals.

As shown in FIG. 2, a noise signal Vs, e.g., a common mode noise signal, may be created on the human skin. The biosignal measuring device may further include a driven right leg circuit (DRL) 60. The DRL 60 extracts the common mode noise signal from the signal output from the biosignal extracting unit 100, inverting-amplifies the extracted common mode noise signal, and feeds the inverting-amplified signal back into the human body.

The signal processor 50 may have various configurations depending on the way the biosignal measuring device is installed (e.g., depending on whether the biosignal measuring device is placed on the human body) or depending on its purposes.

Electrodes, a DRL, and a signal process, known to one of ordinary skill in the art, may be used as the electrodes 10, 20, and 30, the DRL 60, and the signal processor 50, respectively. Known analysis on common mode noise signals that may be created from commercial electricity of a predetermined frequency is applied to a circuit may apply to the occurrence of the noise signal Vs.

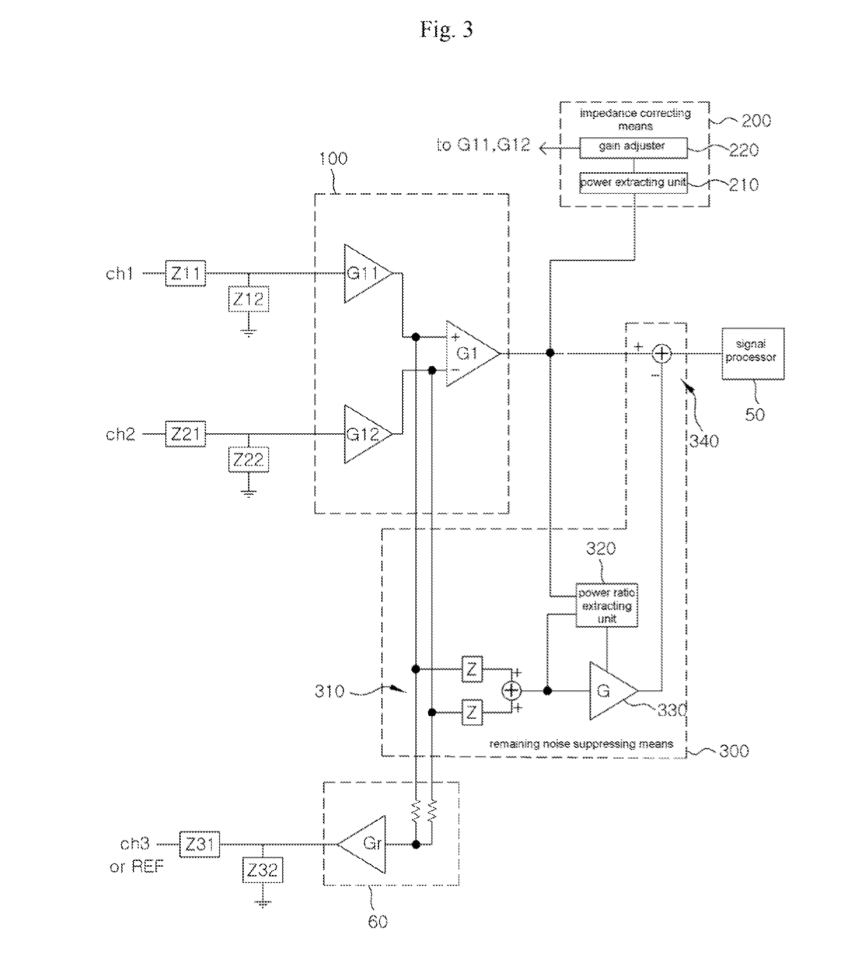

FIG. 3 is an equivalent circuit view illustrating a biosignal measuring device as shown in FIG. 2, according to an embodiment of the present disclosure.

Referring to FIG. 3, the configuration including the electrodes 10 and 20 and conductive wires 11 and 21 of FIG. 2 are illustrated in an electrical equivalent circuit.

For example, the biosignal extracting unit 100 receives bio-potential signals through two channels ch1 and ch2 including the electrodes 10 and 20 and the conductive wires 11 and 21 that may be represented as input impedances.

The respective input impedances of the two channels ch1 and ch2 may include an impedance created where the electrodes 10 and 20 contact the human skin, impedances of the conductive wires 11 and 12, and an impedance of a filter (not shown) or an amplifier (not shown) signal-processing signals transferred through the conductive wires before input to the biosignal extracting unit 100. The impedances may be simply represented as series impedances Z11 and Z21 and parallel impedances Z12 and Z22 as shown in FIG. 3. A conductive wire 31 and a reference electrode 30 connected to the DRL 60 may be represented as a series impedance Z31 and a parallel impedance Z32, respectively, on the output side of the DRL 60.

As such, biosignals detected by the electrodes may be input to the biosignal extracting unit 100, while influenced by the input impedances. According to an embodiment of the present disclosure, the amplifiers G11 and G12, the impedance correcting means 200, and the remaining noise suppressing means 300 may remove influence by any imbalance in input impedance between the two channels ch1 and ch2.

The amplifiers G11 and G12 may be configured so that their amplitude may be adjusted by the impedance correcting means 200. The biosignal extracting unit 100 includes a differential-operating component G11 performing a differential operation. The amplifiers G11 and G12, respectively, are connected to a positive (+) input terminal and negative (−) input terminal of the differential-operating component G1. The amplifiers G11 and G12 may individually amplify bio-potential signals respectively received through the two channels ch1 and ch2 while varying amplitude, before the bio-potential signals are input to the differential-operating component G1 through the positive (+) and negative (−) input terminals.

For example, the amplifiers G11 and G12, each, may be a voltage controlled amplifier (VCA) that varies amplitude depending on the magnitude of a direct current (DC) voltage applied to a control terminal. The impedance correcting means 200 may be configured to apply DC voltages for controlling the amplitude of the amplifiers G11 and G12 to the control terminals of the amplifiers G11 and G12.

A favored environment to obtain a biosignal may be achieved when the impedances are balanced, for example when the series impedances Z11 and Z21 are identical to each other, and the parallel impedances Z12 and Z22 are identical to each other. When the impedances are balanced, common mode noise signals input to the biosignal extracting unit 100 are the same in magnitude as each other, and thus, the common mode noise signals may be suppressed by a differential operation.

However, even a slight motion of the human body might vary the series impedances Z11 and Z21 due to variations in the contact resistance between the electrodes and the human body, resulting in an impedance imbalance between the series impedances Z11 and Z21 even though the impedance balance is made before measuring the biosignal. An impedance imbalance may also arise due to errors in characteristics of the components constituting each channel ch1 and ch2, as well as the contact resistance between the electrodes and the human body.

Influence by the common noise signals due to the impedance imbalance is described below.

For simplicity of calculation, assume that each series impedance Z11 and Z21 consists of only a real-number resistance component, and each parallel impedance Z12 and Z22 only a real-number capacitance component. For example, assume that, in the impedance-balanced state, each series impedance Z11 and Z21 is 100Ω, each parallel impedance Z12 and Z22 is 1000Ω, and the amplitude of the differential-operating component G1 is 1000. When the biosignal created from the human body is 1 mV, the common noise signal may reach nearly a few hundreds or thousands of times the biosignal, e.g., 1000 mV. When such an impedance imbalance occurs where one series impedance, e.g., Z21, varies from 100Ω to 110Ω, undesired signals mixed with a substantial level of common mode noise may be measured as shown in Table 1 below.

TABLE 1

| | | | | Remaining common |
|---|---|---|---|---|
| | | | | mode noise signal after |
| | Series | Parallel | Common mode noise signal per | differential |
| Channel | impedance | impedance | channel | amplification |
| ch1 | Z11 = | Z12 = | 1000 mV * 1000/(100 + 1000) = | (909.091 − |
| | 100 Ω | 1000 Ω | 909.091 mV | 900.901) * 1000 = |
| ch2 | Z21 = | Z22 = | 1000 mV * 1000/(110 + 1000) = | 8190 mV |
| | 100 + 10 = | 1000 Ω | 900.901 mV | |
| | 110 Ω | | | |

As evident from Table 1, when a series impedance, e.g., Z21, is increased by 10%, resulting in an impedance imbalance, the difference in magnitude between the common mode noise signals input through the channels ch1 and ch2 to the biosignal extracting unit 100 occurs between the channels ch1 and ch2, and when differentially operated, 8190 mV of common mode noise ends up being included in 1V (1000 mV) of biosignal. As such, a tiny difference in impedance between the channels ch1 and ch2 may render it difficult to obtain a desired biosignal waveform.

According to an embodiment of the present disclosure, the amplifiers G11 and G12 are provided in the biosignal extracting unit 100, amplifying signals respectively input through the channels ch1 and ch2. The gain of one G12 of the amplifiers G11 and G12 may be finely adjusted and amplified, thus producing a common mode noise-suppressed signal where the biosignal to be measured is more noticeable.

TABLE 2

| | | | | Per-channel | Remaining |
|---|---|---|---|---|---|
| | | | | common mode | common |
| | | | | noise signal after | mode noise |
| | | | | amplification by | signal after |
| | Series | Parallel | Common mode noise | amplifier (G11, | differential |
| Channel | impedance | impedance | signal per channel | G12) | amplification |
| ch1 | Z11 = | Z12 = | 1000 mV * | Gain of G11 = 1 | (909.091 − |
| | 100 Ω | 1000 Ω | 1000/(100 + 1000) = | 909.091 mv * 1 = | 909.009) * |
| | | | 909.091 mV | 909.091 mV | 1000 = |
| ch2 | Z21 = | Z22 = | 1000 mV * | Gain of | 81.9 mV |
| | 100 + 10 = | 1000 Ω | 1000/(110 + 1000) = | G12 = 1.009 | |
| | 110 Ω | | 900.901 mV | 900.901 mV * | |
| | | | | 1.009 = | |
| | | | | 909.009 mV | |

As shown in Table 2 above, even when an impedance imbalance between the channels ch1 and ch2 occurs, the common mode noise signals respectively input through the two channels ch1 and ch2 to the differential-operating component G1 may be rendered substantially the same by finely adjusting and amplifying the gain of one G12 of the amplifiers G11 and G12. Accordingly, after differentially operated, 1V (1000 mV) of biosignal includes substantially 81.9 mV of common mode noise signal. Therefore, a signal waveform including common mode noise signals remarkably reduced as compared with the biosignal may be obtained.

In the example described above in connection with Table 2, the amplifier G12 only is gain-adjusted for ease of description. However, embodiments of the present disclosure are not limited thereto. Alternatively, a variation in power of the biosignal obtained after the differential operation may be substantially removed by increasing the gain of one (e.g., G12) of the amplifiers G11 and G12 while reducing the gain of the other (e.g., G11).

As such, the amplitude of the amplifiers G11 and G12 in the biosignal extracting unit 100 may be adjusted by the impedance correcting means 200, compensating for an impedance imbalance between the channels ch1 and ch2.

The impedance correcting means 200 includes a power extracting unit 210 obtaining the power of common mode noise signals in a signal obtained by performing a differential operation by the biosignal extracting unit 100 and a gain adjuster 220 monitoring variations in power of common mode noise signals while finely varying the amplitude of the amplifiers G11 and G12 in the biosignal extracting unit 100, tracing an amplitude at which the power of the common mode noise signals is reduced, and reducing the amplitude of the amplifiers G11 and G12.

For example, the gain adjuster 220 may increase the amplitude of one of the amplifiers G11 and G12 and decrease the amplitude of the other of the amplifiers G11 and G12.

For example, the gain adjuster 220 may gradually increase the amplitude of one of the amplifiers G11 and G12 while gradually decreasing the amplitude of the other of the amplifiers G11 and G12 to discover an amplitude at which the power of common mode noise signals is reduced but the power of biosignal remains substantially constants. For example, the gain adjuster 220 may, after setting a large variation in amplitude at an early stage of its operation, gradually reducing the variation in amplitude and repeats the amplitude adjustment until the power of common mode noise signals converges into a value less than a predetermined value. If the power of common mode noise signals is increased while the amplitude of one of the amplifiers G11 and G12 is increased and the amplitude of the other is reduced, varying the amplitude may be performed in an opposite way to discover an amplitude at which the power of the common mode noise signals is reduced. For example, if the power of common mode noise signals is increased while the amplitude of the amplifier G11 is increased and the amplitude of the other G12 is reduced, an amplitude at which the power of the common mode noise signals is reduced may be discovered by reducing the amplitude of the amplifier G11 while increasing the amplitude of the amplifier G12.

The power extracting unit 210 may consider the power of a commercial electricity frequency component as the power of a common mode noise signal. The power extracting unit 210 may extract a commercial electricity frequency component from a signal obtained by the differential operation of the biosignal extracting unit 100, and the gain adjuster 220 may finely adjust the amplifiers G11 and G12, monitor the power of the commercial electricity frequency component, and trace an amplitude at which the power of the commercial electricity frequency component is reduced. This is why upon extracting a biosignal, noise signals may be introduced in the biosignal due to the commercial electricity. For example, a 60 Hz component in Korea and a 50 Hz in Europe may be considered as common mode noise signals.

According to an embodiment of the present disclosure, a predetermined common mode noise signal may be intentionally applied to the human body through the reference electrode 30 for the DRL 60 negatively feeding common mode noise signals back to the human body or a third electrode, and the common mode noise signals may be monitored to adjust the amplitude of the amplifiers G11 and G12.

The predetermined common mode noise signal may be a signal of frequency or a particular pattern. When having a particular frequency, the predetermined common mode noise signal may be extracted using a frequency modulator or filter such as using Fast Fourier Transform (FFT), and when having a particular pattern, the predetermined common mode noise signal may be extracted and monitored using a correlation analysis scheme.

A signal generator (not shown) for generating a signal of frequency or pattern predetermined by the user, the reference electrode 30 or a third electrode for applying a signal generated by the signal generator to the human body may be provided to use the predetermined common mode noise signal.

When the signal of a particular frequency is applied to the human body, the power extracting unit 210 may extract the power of the signal with the predetermined frequency from a signal obtained by a differential operation of the biosignal extracting unit 100, and the gain adjuster 220 monitors the power of the signal with the predetermined frequency while finely adjusting the amplitude of the amplifiers G11 and G12 and traces an amplitude at which the power of the signal with the predetermined frequency is reduced.

When a signal of a particular pattern is applied to the human body, the signal is sensed by an electrode, signal-processed by the biosignal extracting unit 100, and is then output. The power extracting unit 210 obtains a correlation between the signal obtained by the differential operation of the biosignal extracting unit 100 and the signal of the particular pattern, applied to the human body, and the gain adjuster 220 monitors the correlation resulted by finely adjusting the amplitude of the amplifiers G11 and G12 and discovers an amplitude at which the correlation is reduced. Adjusting the amplitude so that the correlation is reduced may mean reducing the power of noise.

Alternatively, the amplitude may be adjusted so that the power of a signal obtained by the differential operation of the biosignal extracting unit 100 is reduced, without considering a signal of a particular frequency as a common mode noise signal. This alternative method may apply, e.g., in the circumstance where a significant impedance imbalance occurs and thus the common mode noise signal in a signal obtained by the differential operation of the biosignal extracting unit 100 is much larger in magnitude than the biosignal. For example, when the common mode noise signals is at a very high level, the impedance imbalance may be, at least partially, addressed by adjusting amplitude so that the power of a signal obtained by the differential operation of the biosignal extracting unit 100, without extracting an signal of frequency.

For example, the impedance correcting means 200, a power ratio extracting unit 320 of the remaining noise suppressing means 300, and the signal processor 50 may be configured in a microprocessor performing digital data processing. In this case, an analog-to-digital (AD) converter may be used. To adjust the amplitude of the amplifiers G11 and G12 by the microprocessor, an amplitude-adjusted signal may be converted by a digital-to-analog (D/A) converter, and the D/A converted signal may be input to the control terminal of each amplifier G11 and G12. The differential-operating component G1 of the biosignal extracting unit 100 may be a differential amplifier or an A/D converter obtaining a digital signal by the differential operation. As the above-described AD converter and D/A converter, a known A/D converter and D/A converter may be put to use.

Despite the noise suppression by the amplifiers G11 and G12 together with the impedance correcting means 200, the common mode noise signals might not be completely removed as shown in Table 2 above in case fine adjustment of the amplifiers G11 and G12 to suppress common mode noise signals may be limited. The common mode noise signals that may be left after the suppressing operation by the amplifiers G11 and G12 and the impedance correcting means 200 may be further suppressed by the remaining noise suppressing means 300 as described below.

The remaining noise suppressing means 300 extracts common mode noise signals from a signal obtained by the differential operation of the biosignal extracting unit 100, normalizes the common mode noise signals, and subtracts the common mode noise signals from the signal obtained by the differential operation of the biosignal extracting unit 100. Accordingly, the remaining noise suppressing means 300 may produce a signal where the remaining common mode noise signals not suppressed by the amplifiers G11 and G12 and the impedance correcting means 200 has been sufficiently removed.

The remaining noise suppressing means 300 includes a common mode noise extracting unit 310 that sums signals respectively amplified by the two amplifiers G11 and G12 of the biosignal extracting unit 100 to obtain a common mode noise signal, a power ratio extracting unit 320 that obtains a ratio in power of a signal obtained by the differential operation of the biosignal extracting unit 100 to the obtained common mode noise signal, a common mode noise normalizing unit 330 that multiplies the obtained common mode noise signal by the obtained power ratio to normalize the common mode noise signal, and a subtractor 340 that subtracts the normalized common mode noise signal from the signal obtained by the differential operation of the biosignal extracting unit 100.

The power ratio extracting unit 320 obtains the power of the respective commercial electricity frequency components of the common mode noise signal obtained by the common mode noise extracting unit 310 and the signal obtained by the differential operation of the biosignal extracting unit 100 using a processor used in the power extracting unit 210 to thus obtain the power of the common mode noise signals for acquiring the power ratio. This may be performed because the signal obtained by summing the signals respectively amplified by the two amplifiers G11 and G12 of the biosignal extracting unit 100 may contain noise signals other than the commercial electricity frequency component or biosignal.

According to an embodiment of the present disclosure, a predetermined frequency of signal may be applied to an electrode other than the electrodes for measuring the bio-potential signals, and the power of predetermined frequency components may be respectively obtained from the common mode noise signal obtained by the common mode noise extracting unit 310 and the signal obtained by the differential operation of the biosignal extracting unit 100, thus acquiring the power ratio.

Power of particular frequency (a commercial electricity frequency component or the predetermined frequency) may be extracted through, e.g., Fast Fourier Transform (FFT).

In case a signal of pattern is applied to the human body, a correlation between the signal of pattern and the common mode noise signal obtained by the common mode noise extracting unit 310 and a correlation between the signal of pattern and the signal obtained by the differential operation of the biosignal extracting unit 100 may be obtained, and a ratio of the obtained correlations may be used as the power ratio.

As such, the common mode noise signals left by the operation of the impedance correcting means 200 may be further suppressed by the operation of the remaining noise suppressing means 300, thus producing a signal with common mode noise signals sufficiently removed.

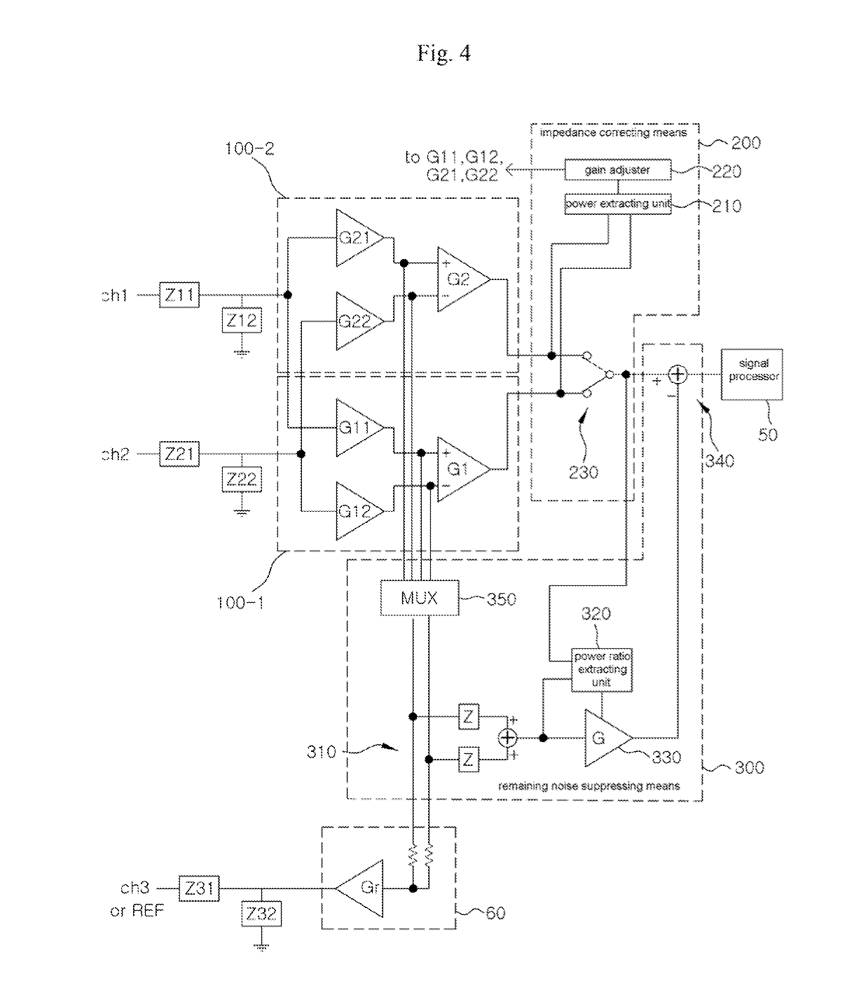

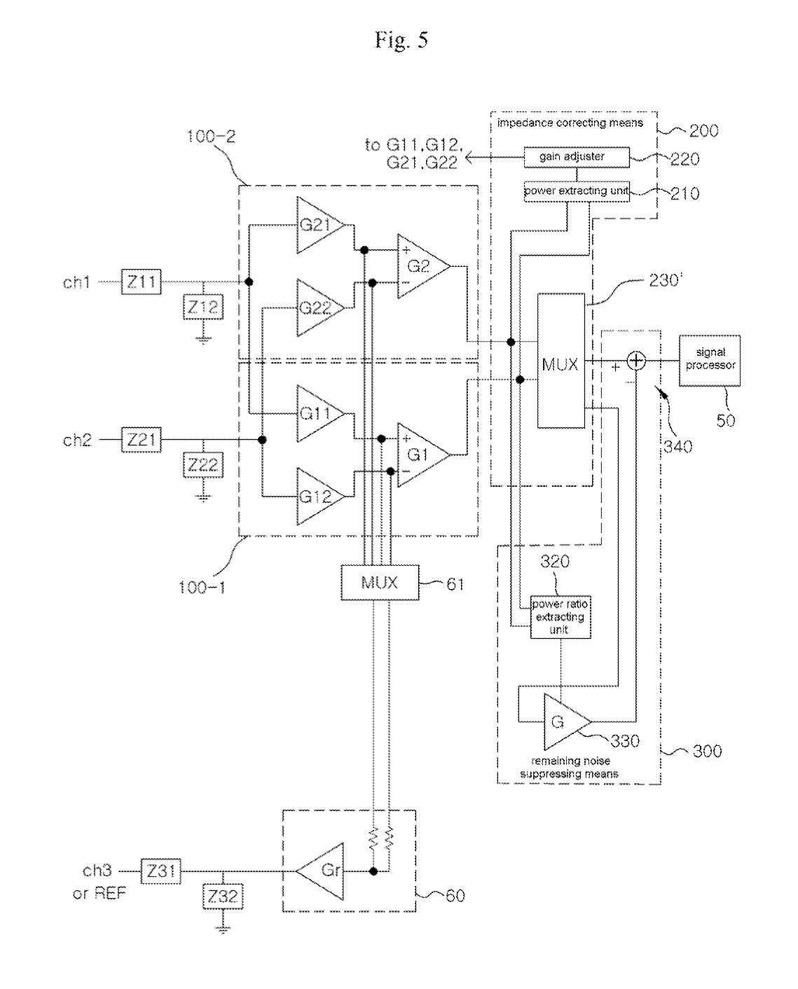

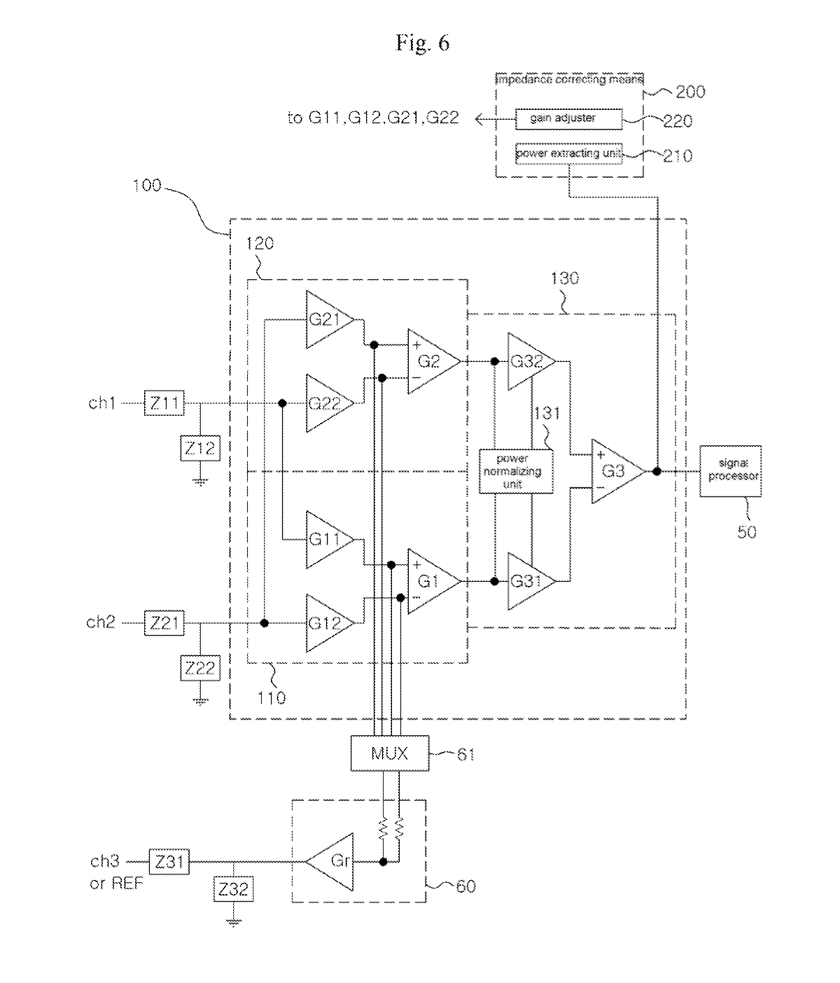

According to an embodiment of the present disclosure, the bio signal extracting unit 100 includes two detecting means connected in parallel with each other and respectively connected with two channels. Each detecting means may be a component that amplifies bio potential signals respectively received through the two channels through respective amplifiers and differentially operates the amplified bio potential signals through a differential operator. In the embodiment described above in connection with FIG. 3, the bio signal extracting unit 100 may be a detecting means. In embodiments as will be described below in connection with FIGS. 4 and 5, first and second bio signal extracting units 100-1 and 100-2 may be two detecting means, respectively. In an embodiment as will be described below in connection with FIG. 6, first and second pre-processors 110 and 120 may be two detecting means, respectively.

According to an embodiment of the present disclosure, a biosignal measuring device may include a means to selectively adopt signals respectively output from the two detecting means to obtain a common mode noise-suppressed signal. In the embodiments as will be described below in connection with FIGS. 4 and 5, a selecting means, e.g., a switch or multiplexer (MUX), may be further provided to alternately select the two detecting means and to finally select one of the two detecting means. In the embodiment as will be described below in connection with FIG. 6, the two detecting means both may be adopted, and there may be further provided a means to process signals respectively output from the two detecting means to obtain a biosignal.

FIG. 4 is a circuit view illustrating a biosignal measuring device according to an embodiment of the present disclosure.

Referring to FIG. 4, the biosignal measuring device includes two biosignal extracting units 100-1 and 100-2 also denoted a first biosignal extracting unit 100-1 and a second biosignal extracting unit 100-2, respectively. The first biosignal extracting unit 100-1 and the second biosignal extracting unit 100-2 are connected in parallel with each other and are respectively connected to two channels ch1 and ch2, respectively.

The power extracting unit 210 of the impedance correcting means 200 obtains differential-operated signals respectively by the first biosignal extracting unit 100-1 and the second biosignal extracting unit 100-2 and thus obtains the power of common mode noise signals.

The gain adjuster 220 repeats the process of adjusting the amplitude of the amplifiers of one of the first biosignal extracting unit 100-1 and the second biosignal extracting unit 100-2, and when the common mode noise signal obtained by the one is smaller than the common mode noise signal obtained by the other, adjusting the amplitude of the other while leaving the one in the amplitude-adjusted state, and when the power of the common mode noise signal reaches a predetermined convergent condition, selects the biosignal extracting unit producing the smaller common mode noise signal power by a switch 230 to transfer the biosignal to the remaining noise suppressing means 300. The switch 230 may be a multiplexer (MUX).

The predetermined condition may include a condition where the common mode noise signal is smaller than a predetermined value or a condition where, despite the amplitude adjustment, the ratio or degree at which the common mode noise signal power is reduced is slow and is lower than a predetermined value.

For example, while the amplitude of the amplifiers G11 and G12 of the first biosignal extracting unit 100-1 remains the same as the amplitude of the amplifiers G21 and G22 of the second biosignal extracting unit 100-2, comparison in power is performed between the common mode noise signals respectively obtained by the respective differential operations of the first biosignal extracting unit 100-1 and the second biosignal extracting unit 100-2. For example, when the common mode noise signals obtained by the first biosignal extracting unit 100-1 is smaller than the common mode noise signals obtained by the second biosignal extracting unit 100-2, the amplitude of the amplifiers G21 and G22 of the second biosignal extracting unit 100-2 is varied, and it is monitored whether the common mode noise signals obtained by the second biosignal extracting unit 100-2 is smaller than the common mode noise signals obtained by the first biosignal extracting unit 100-1. The variation in amplitude may be performed by increasing the amplitude of one of the two amplifiers G21 and G22 and reducing the amplitude of the other of the two amplifiers G21 and G22, for example.

The amplitude of the amplifiers G21 and G22 of the second biosignal extracting unit 100-2 is varied, and when the common mode noise signals obtained by the second biosignal extracting unit 100-2 is smaller than the common mode noise signals obtained by the first biosignal extracting unit 100-1, the amplitude of the amplifiers G11 and G12 of the first biosignal extracting unit 100-1 is varied while the varied amplitude remains substantially the same, and it is monitored whether the common mode noise signals obtained by the first biosignal extracting unit 100-1 is smaller than the common mode noise signals obtained by the second biosignal extracting unit 100-2. The variation in the amplitude of the amplifiers G11 and G12 of the first biosignal extracting unit 100-1 may be performed after the amplitude of the amplifiers G11 and G12 of the first biosignal extracting unit 100-1 has been adjusted to the adjusted amplitude of the amplifiers G21 and G22 of the second biosignal extracting unit 100-2. As such, a variation in amplitude of one of the biosignal extracting units may be performed reflecting the amplitude of the other of the biosignal extracting units.

When the power of common mode noise signals reaches the predetermined convergent condition while the amplifiers G11 and G12 of the first biosignal extracting unit 100-1 and the amplifiers G21 and G22 of the second biosignal extracting unit 100-2 are alternately adjusted, the biosignal obtained by the biosignal extracting unit producing a relatively smaller common mode noise signal power among the biosignal extracting units 100-1 and 100-2 is selected by the switch 230. If the impedance difference is varied, the above process may be repeated to reselect one of the biosignal extracting units 100-1 and 100-2 by the switch 230.

The remaining noise suppressing means 300 includes a multiplexer (MUX) 350 that receives signals respectively amplified by the amplifiers of the first biosignal extracting unit 100-1 and the second biosignal extracting unit 100-2 and selects one of the received signals as a signal for obtaining a common mode noise signal. After one of the first biosignal extracting unit 100-1 and the second biosignal extracting unit 100-2 is selected by the switch 230 of the impedance correcting means 200, the MUX 350 may receive a signal from the biosignal extracting unit selected by the switch 230 and obtain a common mode noise signal from the received signal. The operation of the power ratio extracting unit 320, the common mode noise normalizing unit 330, and the subtractor 340 using the signal selected by the MUX 350 may be substantially the same as the operation described above in connection with FIG. 3.

The biosignal measuring device shown in FIG. 4 includes a DRL 60. Accordingly, a common mode noise signal to be negatively fed back to the human body may be obtained from the signal selected by the MUX 350. A separate MUX may be provided to receive a signal from the biosignal extracting unit selected by the switch 230 and obtain a common mode noise signal to be negatively fed back to the human body.

FIG. 5 is a circuit view illustrating a biosignal measuring device according to an embodiment of the present disclosure.

Referring to FIG. 5, similar to the embodiment shown in FIG. 4, the biosignal measuring device includes two biosignal extracting units 100-1 and 100-2 and adjusts one of the biosignal extracting units, which produces a relatively smaller common mode noise signal by alternately adjusting amplitude using the impedance correcting means 200. The biosignal measuring device shown in FIG. 5 includes a multiplexer (MUX) 230′ to select a biosignal extracting unit, unlike the embodiment shown in FIG. 4 using the switch 230.

In this embodiment, the remaining noise suppressing means 300 does not include a common mode noise extracting unit 310 unlike the embodiments described in connection with FIGS. 3 and 4, and the remaining noise suppressing means 300 obtains a common mode noise signal using the biosignal extracting unit that is not selected by the MUX 230′.

For example, similar to the embodiment described in connection with FIG. 4, the gain adjuster 220 of the impedance correcting means 200 alternately adjusts the amplitude of the amplifiers of the biosignal extracting units 100-1 and 100-2, and after selecting the biosignal extracting unit producing a relatively smaller common mode noise signals under the predetermined convergent condition, controls one of the two amplifiers of the unselected biosignal extracting unit to perform inverting-amplification or cuts off the output of amplified signals (substantially the same as allowing the amplitude to be 0). In the signal obtained by the differential operation of the unselected biosignal extracting unit, the common mode noise signals then prevails over the biosignal to be measured. In other words, the common mode noise signals is significantly large as compared with the biosignal.

The MUX 230′ performs a switching operation to select the biosignal extracting unit producing a relatively smaller common mode noise signals while transferring the output from the unselected biosignal extracting unit to the common mode noise normalizing unit 330 of the remaining noise suppressing means 300.

The power ratio extracting unit 320 obtains the power of common mode noise signals from signals respectively output from the biosignal extracting units 100-1 and 100-2, obtains a ratio in power of the common mode noise signals included in the signal (signal with the common mode noise signals suppressed by compensating for the impedance imbalance between the channels) output from the biosignal extracting unit selected by the MUX 230′ to the common mode noise signals included (signal with a significantly large common mode noise signals than the biosignal) output from the biosignal extracting unit not selected by the MUX 230′, and reflects the obtained power ratio to the common mode noise normalizing unit 330.

Accordingly, a normalized common mode noise signals may be obtained by the common mode noise normalizing unit 330, and the common mode noise signals remaining in the signal output from the biosignal extracting unit selected by the MUX 230′ may be suppressed by the subtractor 340. In the embodiment shown in FIG. 5, the operation of the power ratio extracting unit 320, the common mode noise normalizing unit 330, and the subtractor 340 may be substantially the same as the operation described above in connection with FIGS. 3 and 4.

The biosignal measuring device shown in FIG. 5 includes a DRL 60. Accordingly, a separate multiplexer (MUX) 61 may be provided selecting one of the biosignal extracting units 100-1 and 1002 to obtain a common mode noise signal.

The remaining noise suppressing means 300 shown in FIG. 5 may be further simplified as compared with the remaining noise suppressing means 300 shown in FIG. 4. The impedance correcting means 200 and the remaining noise suppressing means 300 may be configured in a single microprocessor, so that the processor to obtain common mode noise signal power, as used in the power extracting unit 210, may be used in the power ratio extracting unit 320.

FIG. 6 is a circuit view illustrating a biosignal measuring device according to an embodiment of the present disclosure. The biosignal measuring device includes a biosignal extracting unit 100. The biosignal extracting unit 100 includes a first pre-processor 110 and a second pre-processor 120, each including amplifiers, and a differential operating unit 130. The first pre-processor 110 and the second pre-processor 120, respectively, amplify the respective bio-potential signals from two channels ch1 and ch2 each including electrodes and conductive wires, using their amplifiers, and perform a differential operation on the amplified signals. The differential operating unit 130 performs power normalization on the signals respectively and independently pre-processed by the first pre-processor 110 and the second pre-processor 120 so that the common mode noise signals are of the same power and then performs a differential operation on the power-normalized signals to produce a common mode noise signal-suppressed biosignal.

The impedance correcting means 200 may trace and adjust amplifier by substantially simultaneously adjusting the amplitude of the amplifiers included in the two pre-processors 110 and 120, so that the power of common mode noise signals included in the biosignal obtained by the differential operator 130 is reduced.

The first pre-processor 110 and the second pre-processor 120 are connected in parallel with each other and are connected to the first and second channels ch1 and ch2, respectively. Each channel is connected to a positive (+) input terminal of a differential operator of one of the first pre-processor 110 and the second pre-processor 120 and a negative (−) input terminal of a differential operator of the other of the first pre-processor 110 and the second pre-processor 120.

For example, the first pre-processor 110 and the second pre-processor 120, respectively, include differential operators G1 and G2 each having a positive (+) input terminal and a negative (−) input terminal. The differential operators G1 and G2 each perform a differential operation on signals input through the positive (+) input terminal and the negative (−) input terminal. One channel ch1 is branched and connects to each of the first pre-processor 110 and the second pre-processor 120. The channel ch1 is connected to the positive (+) input terminal of the differential operator G11 via the amplifier G11 in the first pre-processor 110 and to the negative (−) input terminal of the differential operator G2 via the amplifier G22 in the second pre-processor 120.

The other channel ch2 is branched and connects to each of the first pre-processor 110 and the second pre-processor 120. The channel ch2 is connected to the negative (−) input terminal of the differential operator G1 via the amplifier G12 in the first pre-processor 110 and to the positive (+) input terminal of the differential operator G2 via the amplifier G21 in the second pre-processor 120.

In other words, the polarity of the input terminal of the differential operator G1 of the first pre-processor 110, through which a channel signal is input, is opposite the polarity of the input terminal of the differential operator G2 of the second pre-processor 120.

By making such connections between the first pre-processor 110 and the second pre-processor 120 and the channels ch1 and ch2, when noise components of the same phase in bio-potential signals coming through the two channels ch1 and ch2 pass through the first pre-processor 110 and the second pre-processor 120, the noise component pre-processed by the first pre-processor 110 and the noise component pre-processed by the second pre-processor 120 may become the same phase or reverse-phase depending on the amplitude of the amplifiers G11, G12, G21, and G22.

If one of the input terminals of each differential operator G1 and G2 is previously designated for its polarity, and a ratio between the amplitude of the amplifiers connected to the input terminal of designated polarity and the amplitude of the amplifiers connected to the other input terminal is rendered higher than a common mode noise signal power ratio, the noise components included in the signals pre-processed by the first pre-processor 110 and the second pre-processor 120 become the same phase.

An example is described with reference to FIG. 6. For example, if a ratio of the amplitude of the amplifier G12 to the amplitude of the amplifier G11 in the first pre-processor 110 and a ratio of the amplitude of the amplifier G22 to the amplitude of the amplifier G21 in the second pre-processor 120 are larger than the power ratio of common mode noise signals coming through the channels ch1 and ch2, the noise components included in the signals pre-processed by the first pre-processor 110 and the second pre-processor 120 become the same phase.

In this case, when the signals pre-processed by the first pre-processor 110 and the second pre-processor 120 are differential-operated by the differential operating unit 130, the common mode noise signals are suppressed.

Accordingly, if the impedance correcting means 200 adjusts the amplitude of the amplifiers of the first pre-processor 110 and the second pre-processor 120 so that the common mode noise signals in the signals obtained by the differential operating unit 130 are reduced, the amplitude of the amplifiers converges into a ratio at which the common mode noise signals are suppressed.

According to an embodiment of the present disclosure, one of the input terminals of the differential operator in each pre-processor 110 and 120 is previously designated for polarity, and the amplitude of amplifiers connected to the input terminal of the designated polarity is adjusted to be larger than the amplitude of amplifiers connected to the input terminal of the other polarity, quickly tracing the amplitude of the amplifiers included in the first pre-processor 110 and the second pre-processor 120. For example, the process of tracing amplitude is performed by finely adjusting amplitude under the condition where the amplitude of the amplifiers G11 and G21 connected to the positive (+) input terminals of the differential operators G1 and G2 is rendered to be larger than the amplitude of the amplifiers G12 and G22 connected to the negative (−) input terminals of the differential operators G1 and G2.

According to the arrangement of the electrodes 10 and 20 (positions where the electrodes 10 and 20 are attached onto the human body) to obtain biosignals in the process of tracing amplitude as described above, even when the reverse-phase biosignal components introduced through the two channels ch1 and ch2 pass through the first pre-processor 110 and the second pre-processor 120, the biosignal component pre-processed by the first pre-processor 110 and the biosignal component pre-processed by the second pre-processor 120 become reverse-phase.

Accordingly, the reverse-phase biosignals included in the pre-processed signals, even when differentially operated by the differential operating unit 130, are left with sufficient power without suppressed.

Referring to FIG. 6, the differential operating unit 130 amplifies signals respectively pre-processed by the first pre-processor 110 and the second pre-processor 120 using amplifiers G31 and G32, differentially operates the amplified signals using a differential operator G3, and adjusts the amplitude of the amplifiers G31 and G32 using a power normalizing unit 131.

For example, the power normalizing unit 131 extracts the power of common mode noise signals from signals pre-processed by the first pre-processor 110 and the second pre-processor 120 and adjusts the amplitude of the amplifiers G31 and G32 so that the power of the common mode noise signal included in the signal pre-processed by the first pre-processor 110 is the same as the power of the common mode noise signal included in the signal pre-processed by the second pre-processor 120. Alternately, one of the two amplifiers G31 and G32 may be adjusted for amplitude, so that the common mode noise signals are rendered the same in power.

Accordingly, the signals respectively input to the positive (+) input terminals and the negative (−) input terminals of the differential operator G3 have the same power of common mode noise signals. Thus, a common mode noise-suppressed signal, e.g., a signal in which the biosignal to be obtained is sufficiently large and the common mode noise signals is suppressed, may be obtained by the differential operation of the differential operator G3. Since errors may occur in the process of extracting the common mode noise signal power, “the common mode noise signals being of the same power” may mean that the common mode noise signals may be sufficiently suppressed to a desired level by the differential operation of the differential operator G3.

As such, a common mode noise-suppressed biosignal may be obtained even when an impedance imbalance occurs between the channels by adjusting the amplitude of the amplifiers G11 and G12 of the first pre-processor 110 and the amplitude of the amplifiers G21 and G22 of the second pre-processor 120 so that the common mode noise signals in the signal differentially operated by the differential operating unit 130 using the power normalizing unit 131 is minimized. The differential operation is performed after the common mode noise signals are rendered the same in power by the amplifiers G31 and G32 whose amplitude is adjusted by the power normalizing unit 131, a biosignal with the common mode noise signals substantially removed may be obtained. Accordingly, the biosignal measuring device shown in FIG. 6 might not have a remaining noise suppressing means 300 as shown in FIGS. 3 to 5.

The extraction of common mode noise signal power for normalization in the power normalizing unit 131 and the extraction of common mode noise signal power for tracing amplitude in the impedance correcting means 200 may be achieved by any one of the extraction of commercial electricity frequency component, application of a signal with a predetermined frequency and then extraction of the power of the signal, and application of a signal with a predetermined pattern to the human body and then extraction of the power of the signal described above in connection with FIGS. 3 to 5. Upon extracting the power of signal with the predetermined pattern, the impedance correcting means 200 adjusts amplitude so that the correlation is reduced as in the embodiments described above in connection with FIGS. 3 to 5, and the differential operating unit 130 obtains correlations between the signal with the predetermined pattern and signals respectively differentially obtained by the first pre-processor 110 and the second pre-processor 120 and adjusts the amplitude of the amplifiers G31 and G32 so that the correlations are rendered the same.

According to an embodiment of the present disclosure, amplifiers are connected to two channels, respectively, receiving bio-potential signals through two electrodes. The amplitude of the amplifiers is adjusted to reduce the power of common mode noise signals. Accordingly, even when an impedance imbalance occurs between the two channels, common mode noise-suppressed biosignals may be obtained.

According to an embodiment of the present disclosure, when an impedance imbalance occurs between the channels, impedance balancing may be achieved by adjusting the amplitude of the amplifiers before performing a differential operation, rather than by adjusting the impedance between the channels. Accordingly, an embodiment of the present disclosure may quickly respond to frequent variations in the channel impedances due to the motion of human body and produce precise biosignal waveforms.

Any common mode noise signals that may remain after the noise suppression by the amplifiers may be further suppressed by the remaining noise suppressing means, thus enabling acquisition of more precise biosignals.

While the inventive concept has been shown and described with reference to exemplary embodiments thereof, it will be apparent to those of ordinary skill in the art that various changes in form and detail may be made thereto without departing from the spirit and scope of the inventive concept as defined by the following claims.

What is claimed is:

1. A biosignal measuring device, comprising
two channels respectively detecting bio-potential signals from a human body through two electrodes;
a biosignal extracting unit including two detecting means respectively amplifying the bio-potential signals through amplifiers, respectively, differentially operating the amplified bio-potential signals through a differential operator, and selectively adopting respective output signals of the two detecting means to obtain a biosignal; and
to an impedance correcting means adjusting the amplitude of the amplifiers of the two detecting means so that the power of a common mode noise signal caused by an impedance imbalance between the channels and included in the obtained biosignal is reduced to suppress the common mode noise signal.

2. The biosignal measuring device of claim 1, wherein the impedance correcting means alternately selects the two detecting means, adjusts the amplitude of an amplifier of a selected detecting means until the amplitude of the amplifier of the selected detecting means is smaller than the amplitude of an amplifier of an unselected detecting means, and when the power of the common mode noise signal reaches a predetermined convergent condition, adopts, as the biosignal, a signal output from one of the two detecting means producing a relatively smaller common mode noise signal.

3. The biosignal measuring device of claim 1, wherein the impedance correcting means starts to adjust the amplitude of the amplifier of the selected detecting means after the amplitude of the amplifier of the selected detecting means reaches the amplitude of the amplifier of the unselected detecting means.

4. The biosignal measuring device of claim 2, further comprising a remaining noise suppressing means summing signals output from two amplifiers included in one of the two detecting means of the biosignal extracting unit to obtain a common mode noise signal, normalizing the obtained common mode noise signal to the power of the common mode noise signal included in the biosignal obtained by the biosignal extracting unit, and subtracting the normalized common mode noise signal from the biosignal obtained by the biosignal extracting unit to obtain a biosignal where a remaining common mode noise signal has been suppressed.

5. The biosignal measuring device of claim 4, wherein after the impedance correcting means is operated, the remaining noise suppressing means is operated to suppress the remaining common mode noise signal after suppressing the common mode noise signal that occurs due to the impedance imbalance between the channels.

6. The biosignal measuring device of claim 1, wherein one of the channels is connected to an amplifier connected to a negative input terminal of a differential operator of a first detecting means of the two detecting means and an amplifier connected to a positive input terminal of a differential operator of a second detecting means of the two detecting means, and the other of the channels is connected to an amplifier connected to a negative input terminal of the differential operator of the second detecting means of the two detecting means and an amplifier connected to a positive input terminal of the differential operator of the first detecting means of the two detecting means, wherein the biosignal extracting unit includes a differential operating unit differentially operating signals output from the two detecting means to obtain a biosignal, and wherein the impedance correcting means adjusts the respective amplifiers of the two detecting means, respectively connected to the channels, so that the power of a common mode noise signal included in the biosignal obtained by the differential operating unit is reduced.

7. The biosignal measuring device of claim 6, wherein the differential operating unit of the biosignal extracting unit normalizes the signals output from the two detecting means so that common mode noise signals included in the signals output from the two detecting means are substantially the same in power and then differentially operates the normalized output signals to obtain the biosignal.

8. The biosignal measuring device of claim 7, wherein the impedance correcting means previously designates a polarity of one of input terminals of the differential operator of one of the two detecting means and adjusts amplitude so that the amplitude of an amplifier connected to the input terminal of the designated polarity is larger than the amplitude of an amplifier connected to another input terminal of the differential operator.

9. The biosignal measuring device of claim 5, wherein the power of the common mode noise signal includes any one of power of a commercial electricity frequency component, power of a signal with a predetermined frequency applied to the human body, and power of a signal with a predetermined pattern.

10. The biosignal measuring device of claim 8, wherein the power of the common mode noise signal includes any one of power of a commercial electricity frequency component, power of a signal with a predetermined frequency applied to the human body, and power of a signal with a predetermined pattern.

11. A biosignal measuring device, comprising:
a first amplifier connected with a first channel;
a second amplifier connected with a second channel;
a third amplifier configured to differentially operate respective outputs of the first amplifier and the second amplifier to output a noise-suppressed signal; and
an impedance corrector configured to adjust the amplitude of the first amplifier and the second amplifier to reduce a common mode noise signal included in the noise-suppressed-signal.
